(19) United States
(12) Patent Application Publication
(10) Pub. No.: US 2021/0355419 A1
(43) Pub. Date: Nov. 18, 2021

(54) BIOREACTOR SYSTEM FOR CULTIVATING MICROALGAE

(71) Applicant: Sophie's BioNutrients Pte. Ltd., Singapore (SG)

(72) Inventor: Yao-Hsin Wang, Singapore (SG)

(73) Assignee: Sophie's BioNutrients Pte. Ltd., Singapore (SG)

(21) Appl. No.: 15/930,681

(22) Filed: May 13, 2020

(51) Int. Cl. C12M 1/00; C12N 1/12; C12M 1/34

(52) U.S. Cl. CPC C12M 21/02; C12N 1/12; C12N 2500/34; C12M 41/26; C12M 41/06; C12M 31/10

(57) ABSTRACT

A bioreactor system for cultivating microalgae is described. The bioreactor system includes a bioreactor. The bioreactor includes one or more holes. One or more light sources are implanted into each of the one or more holes. A culture media comprising a carbon source is located inside of the bioreactor. A microalgae comprising a photoreceptor sensitive to a region of a visible spectrum is located in the culture media. Each of the one or more light sources produce an irradiance of light including the region of the visible spectrum in a sufficient intensity to transduce the photoreceptor of the microalgae.

### FIELD OF THE EMBODIMENTS

The field of the invention and its embodiments relate to a bioreactor system for cultivating microalgae. In particular, the present invention and its embodiments provide a bioreactor system comprising a bioreactor, where the bioreactor has one or more holes configured to receive one or more light sources therein such that the one or more light sources produce an irradiance of light in a region of a visible spectrum in a sufficient intensity to transduce a photoreceptor of microalgae located in a culture media inside of the bioreactor.

### BACKGROUND OF THE EMBODIMENTS

Algae are photosynthetic organisms that grow in a range of aquatic habitats, including lakes, pounds, rivers, and oceans. Algae can tolerate a wide range of temperatures, salinities, and pH values, as well as differing light intensities. Additionally, algae may also grow alone or in symbiosis with other organisms. Algae may be broadly classified as Rhodophyta (red algae), Phaeophyta (brown algae), or Chlorophyta (green algae). Algae may be further classified by size, as macroalgae (which are multicellular, large-size algae that are visible with the naked eye) or microalgae (which are microscopic, single cells that may be prokaryotic or eukaryotic).

Currently, there are an estimated 300,000 to 1 million species of microalgae in existence. Microalgae has recently attracted considerable interest due to their extensive applications in the renewable energy field, the biopharmaceutical field, and the nutraceutical field. Specifically, microalgae may be a sustainable and economical source of biofuels, bioactive medicinal products, and food ingredients. Moreover, microalgae also have applications in wastewater treatment and atmospheric CO₂ mitigation. Thus, microalgae produces a wide range of bioproducts, including polysaccharides, lipids, pigments, proteins, vitamins, bioactive compounds, and antioxidants.

Although microalgae are feasible sources for bioenergy and biopharmaceuticals in general, some limitations remain. One such limitation to microalgae cultivation is light intensity. Light duration and intensity directly affect the photosynthesis of microalgae. At very low and very high light intensities, microalgae cannot grow efficiently. Higher light intensities will increase a photosynthetic rate of the microalgae to a maximum point, after which it levels off until the photosynthetic rate is balanced by photorespiration and photoinhibition. Photorespiration refers to a process in plant metabolism, where the enzyme, ribulose-1,5-bisphosphate carboxylase/oxygenase (RuBisCO), oxygenates ribulose 1,5-bisphosphate (RuBP), wasting some of the energy produced by photosynthesis. Photoinhibition is light-induced reduction in the photosynthetic capacity of a plant, alga, or cyanobacterium. Essentially, optimal light intensity needs to be determined experimentally in each case to maximize CO₂ assimilation and minimize both photorespiration and photoinhibition. Uniform distribution of light is also needed to avoid photoinhibition.

Another limitation to the growth of microalgae is temperature. Each species of microalgae has its own optimal growth temperature. Increasing a temperature to the optimum range exponentially increases algal growth, but an increase or decrease in the temperature beyond the optimal point retards or even stops algae growth and activity. The optimum temperature range for most algal species is 20-30° C. Growing microalgae cultures at non-optimal temperatures will result in high biomass losses.

A further limitation to microalgae growth involves ensuring the nutritional needs of the microalgae are met. Typically, all strains of microalgae have the following backbone: nitrogen, phosphorus, and carbon (CH_{1.7} O_{0.4} N_{0.15} P0.0094). Some marine microalgae species also require silicon as a macronutrient. Specifically, quantities of the available nitrogen in the culture directly alter cell growth. Nitrogen limitation in the microalgae culture can reduce growth and biomass productivity, however, can increase production of carbohydrates and lipids. As an illustrative example, an optimum concentration of nitrogen for Chlorella vulgaris is 0.5 g/l, at which it produces 3.43 g/l biomass. Moreover, the micronutrients molybdenum (Mo), potassium (K), cobalt (Co), iron (Fe), magnesium (Mg), manganese (Mn), boron (B), and zinc (Zn) are only required in trace amounts, but have been shown to have a strong impact on microalgae growth, as they influence many enzymatic activities in algal cells. Nutrient deficiency greatly affects the microalgae growth rate and results in low biomass.

Thus, numerous limitations currently exist to microalgae growth. One conventional method to address these concerns is to grow microalgae under a heterotrophic growth condition. Techniques have been developed for the large-scale production of aquatic microalgae under heterotrophic growth conditions by utilizing organic carbon instead of light as an energy source. However, this method cannot be applied to all microalgae because only a limited number of microalgae strains can grow in heterotrophic conditions. However, no solution currently available addresses all problems faced for microalgae growth. Thus, a need exists for an improved method to cultivate microalgae.

### Review of Related Technology

KR 101287384 B1 describes a method for cultivating microalgae, which includes cultivating Botryococcus braunii in an optical condition mixing LED light with 640 nm wavelengths and 460 nm wavelengths in a ratio of 5:1.

U.S. Patent Application Publication No. 2018/0371395 A9 describes a bioreactor (such as a fermentation tank) for cultivating microalgae. The reference explains that exposing microalgae cells to light, even in the presence of a fixed carbon source that the cells transport and utilize, can accelerate growth compared to culturing cells in the dark. The reference further teaches that the bioreactor system includes a plurality of light sources operatively coupled to the bioreactor, where the plurality of light sources can include LED lights. The plurality of light sources may also be operable to supply full spectrum or a specific wavelength of artificial light into the bioreactor.

CN 203668359 U describes a fermentation tank provided with an LED illuminating lamp. The fermentation tank comprises a tank cover and a fermentation tank body, where an illumination hole is formed in the tank cover and the LED illuminating lamp is mounted in the illumination hole so that the LED illuminating lamp produces lower heat than a conventional illumination lamp while providing the same illumination effect.

EP 2478089 B1 describes a system that includes: a bioreactor, a culture media located within the bioreactor, a light source, and a microalgae located in the culture media and adapted for heterotrophic growth. The culture media comprises a fixed carbon source. The microalgae is a Botryococcus strain, a Neochloris strain, or a Chlamydomonas strain. The light source may include: natural sunlight collected by a solar energy collector or an artificial light produced by a light emitted diode (LED) or a fluorescent light. In some examples, the light source is operatively coupled to the bioreactor. In other examples, one or more optical fibers are mounted in optically transparent lighting structures.

CN 103773673 B describes a method for culturing microalgae in a cylindrical bioreactor, where the bioreactor includes an illumination source that transmits light into the bioreactor.

Various methods to cultivate microalgae are known in the art. However, their means of operation are substantially different from the present disclosure, as the other inventions fail to solve all the problems taught by the present disclosure. The present invention and its embodiments provide a bioreactor system for cultivating microalgae. In particular, the present invention and its embodiments provide a bioreactor system comprising a bioreactor, where the bioreactor has one or more holes configured to receive one or more light sources therein such that the one or more light sources produce an irradiance of light in a region of a visible spectrum in a sufficient intensity to transduce a photoreceptor of microalgae located in a culture media inside of the bioreactor.

### SUMMARY OF THE EMBODIMENTS

The present invention and its embodiments provide a bioreactor system for cultivating microalgae. In particular, the present invention and its embodiments provide a bioreactor system comprising a bioreactor, where the bioreactor has one or more holes configured to receive one or more light sources therein such that the one or more light sources produce an irradiance of light in a region of a visible spectrum in a sufficient intensity to transduce a photoreceptor of microalgae located in a culture media inside of the bioreactor.

A first embodiment of the instant invention describes a bioreactor system for cultivating microalgae. The bioreactor system comprises a bioreactor. The bioreactor comprises one or more holes. In some examples, the bioreactor is a fermentation tank. Moreover, a culture media comprising a carbon source is located inside the bioreactor. The carbon source may be glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, or molasses, among other examples not explicitly listed herein.

Further, a microalgae comprising a photoreceptor sensitive to a region of a visible spectrum is located in the culture media. According to some examples, the microalgae is of a mixotrophic strain. The mixotrophic strain of the microalgae means that the microalgae is adapted for both autotrophic growth and heterotrophic growth during a time period. In some examples, the microalgae is a Botryococcus sudeticus strain, a Botryococcus strain, a Neochloris oleabundans strain, a Neochloris strain, a Chlamydomonas reinhardtii strain, or a Chlamydomonas strain, among other strains not explicitly listed herein.

The bioreactor system also includes one or more light sources implanted or impregnated into each of the one or more holes of the bioreactor. Each of the one or more light sources produces an irradiance of light in the region of the visible spectrum in a sufficient intensity to transduce the photoreceptor of the microalgae. In some examples, each of the one or more light sources includes an artificial light source. In other examples, the artificial light source is a light-emitting diode (LED).

A second embodiment of the instant invention describes a method executed by a bioreactor system to cultivate a microalgae comprising a photoreceptor sensitive to a region of a visible spectrum under a mixotrophic growth condition. The bioreactor system comprises a bioreactor having one or more light sources implanted into one or more holes of the bioreactor. The method includes: incubating the microalgae under the mixotropic growth condition for a time period sufficient to allow the microalgae to grow. The mixotropic growth condition comprises: a culture media located inside of the bioreactor that comprises a carbon source, and an irradiance of light from the one or more light sources that includes the region of the visible spectrum in a sufficient intensity to transduce the photoreceptor.

The method may further include producing a material from the microalgae. The material may be a polysaccharide, a pigment, a lipid, or a hydrocarbon, among other materials not explicitly listed herein. In other examples, the method may additionally include the process steps of recovering the material; extracting the material; and processing the material to form another material. The other material may include: a fuel, biodiesel, jet fuel, a cosmetic, a pharmaceutical agent, a surfactant, or a renewable diesel, among other materials not explicitly listed herein. In further examples, the method may additionally include modifying one or more of the following: modifying the time period to another time period, modifying a pH level of the microalgae, modifying a wavelength of the irradiance of the light from the one or more light sources (e.g., modifying the light spectrum) onto the microalgae, and/or modifying a feed stock for the microalgae to change a combination of amino acids in the microalgae. By doing so, functional proteins can be created.

A third embodiment of the instant invention describes a method executed by a bioreactor system to manufacture a material from a microalgae. The bioreactor system comprises a bioreactor having one or more light sources implanted or impregnated into one or more holes of the bioreactor. The method includes: providing the microalgae comprising a photoreceptor sensitive to a region of a visible spectrum under a mixotrophic growth condition. The microalgae is capable of producing the material. The method also includes: culturing the microalgae in a media located in the bioreactor. The media comprises a carbon source. The method further includes: applying an irradiance of light from the one or more light sources including the region of the visible spectrum in a sufficient intensity to transduce the photoreceptor; producing the material from the microalgae; recovering the material; and extracting the material.

The method may additionally include processing the material to form another material. The other material may be: a fuel, biodiesel, jet fuel, a cosmetic, a pharmaceutical agent, a surfactant, or a renewable diesel, among other materials not explicitly listed herein. The method may further include modifying one or more of the following: modifying the time period to another time period, modifying a pH level of the microalgae, modifying a wavelength of the irradiance of the light from the one or more light sources (e.g., modifying the light spectrum) onto the microalgae, and/or modifying a feed stock for the microalgae to change a combination of amino acids in the microalgae. By doing so, functional proteins can be created.

In general, the present invention succeeds in conferring the following benefits and objectives.

It is an object of the present invention to provide a bioreactor system for cultivating microalgae.

It is an object of the present invention to provide a bioreactor system for cultivating microalgae of a mixotrophic strain.

It is an object of the present invention to provide a bioreactor system for cultivating microalgae adapted for autotrophic growth and heterotrophic growth during a time period.

It is an object of the present invention to provide a bioreactor system for growing microalgae via both light and fermentation.

It is an object of the present invention to provide a bioreactor system comprising a bioreactor, where the bioreactor has one or more holes configured to receive one or more light sources therein such that the one or more light sources produce an irradiance of light in a region of a visible spectrum in a sufficient intensity to transduce a photoreceptor of microalgae located in a culture media inside of the bioreactor.

### BRIEF DESCRIPTION OF THE DRAWINGS

FIG. 1 depicts a schematic block diagram of a process to create biomass from microalgae, according to at least some embodiments described herein.

FIG. 2 depicts a perspective view of an improved bioreactor for cultivating microalgae, according to at least some embodiments described herein.

FIG. 3 depicts another perspective view of an improved bioreactor for cultivating microalgae, according to at least some embodiments described herein.

FIG. 4 depicts another perspective view of an improved bioreactor for cultivating microalgae, according to at least some embodiments described herein.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As described herein, a “bioreactor” is an enclosure or partial enclosure, in which cells are cultured, and optionally in suspension.

As described herein, a “photobioreactor” refers to a container, at least part of which is at least partially transparent or partially open, thereby allowing light to pass through, in which one or more microalgae cells are cultured.

As described herein, a “biodiesel” is a biologically-produced fatty acid alkyl ester suitable for use as a fuel in a diesel engine.

As described herein, a “biomass” refers to a material produced by growth and/or propagation of cells. Biomass may contain cells and/or intracellular contents, as well as extracellular material.

As described herein, “extracellular material” may include, but is not limited to, compounds secreted by a cell.

As defined herein, an “autotroph” refers to an organism that is capable of synthesizing its own food from inorganic substances, using light or chemical energy.

As defined herein, a “heterotroph” refers to an organism that cannot synthesize its own food and is dependent on complex organic substances for nutrition.

As defined herein, a “mixotrophic strain” is defined as a strain of an organism that allows it to be both autotrophic and heterotrophic at the same time.

As defined herein, a “hydrocarbon” refers to: (a) a molecule containing only hydrogen and carbon atoms, where the carbon atoms are covalently linked to form a linear, branched, cyclic, or partially cyclic backbone to which the hydrogen atoms are attached; or (b) a molecule that only primarily contains hydrogen and carbon atoms and that can be converted to contain only hydrogen and carbon atoms by one to four chemical reactions.

As defined herein, a “lipid” refers to a class of hydrocarbons that are soluble in nonpolar solvents and are relatively or completely insoluble in water.

As defined herein, a “microalgae” refers to a eukaryotic microbial organism that contains a chloroplast, and optionally, that is capable of performing photosynthesis, or a prokaryotic microbial organism capable of performing photosynthesis.

As defined herein, a “feed stock” refers to what kind of food waste one uses to feed microalgae. Different feed stocks include differing nitrogen and carbon sources.

As defined herein, a “polysaccharide” refers to a carbohydrate made up of monosaccharides joined together by glycosidic linkages.

As defined herein, the “Benson-Calvin cycle” is a set of chemical reactions that take place in chloroplasts during photosynthesis. To be more specific, carbon fixation produces an intermediate product that is then converted into the final carbohydrate products. The carbon skeletons that are produced by photosynthesis are then used in a variety of processes to form other organic compounds.

As defined herein, “fermentation” refers a metabolic process that produces chemical changes in organic substrates through the action of enzymes. In the context of food production, “fermentation” may refer to any process in which the activity of microorganisms brings about a desirable change to a foodstuff or beverage. In microorganisms, fermentation is the primary means of producing adenosine triphosphate (ATP) by the degradation of organic nutrients anaerobically. As an example, fermentation may be used to produce alcoholic beverages, such as wine and beer.

As defined herein, “axenic” refers to a culture of an organism that is free from contamination by other living organisms.

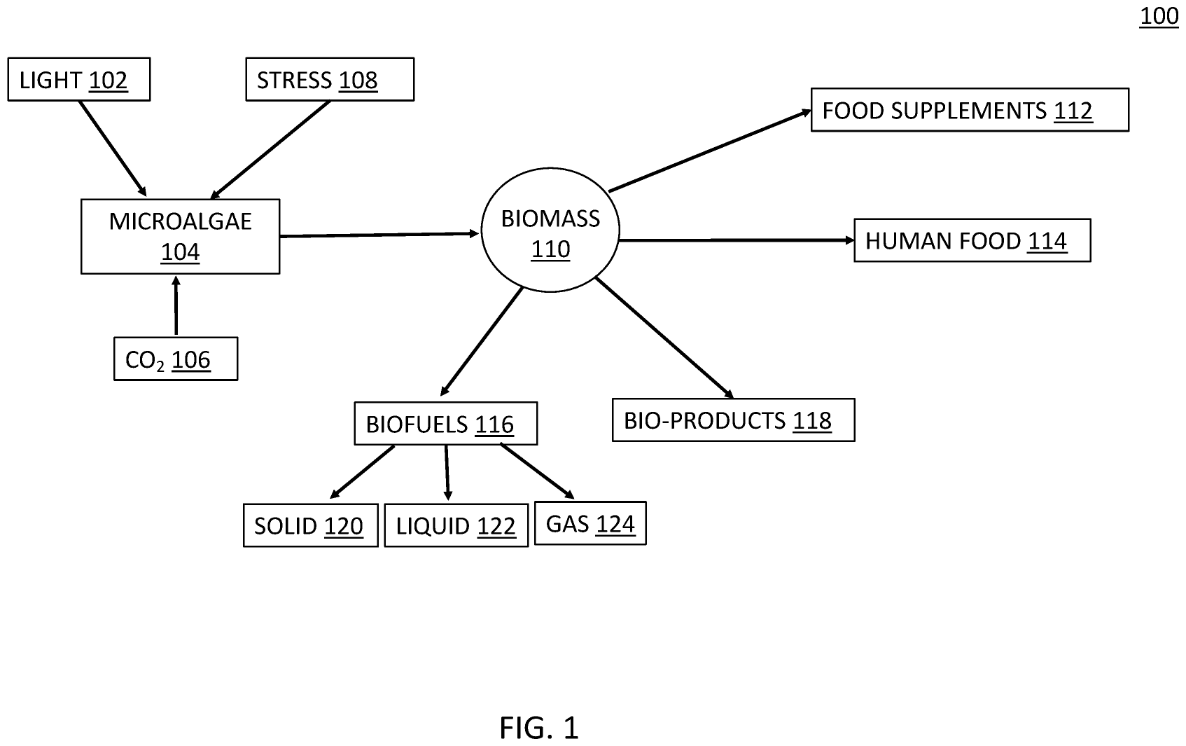

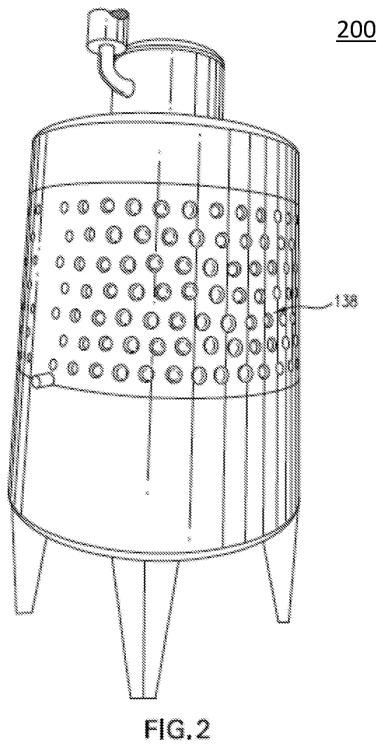

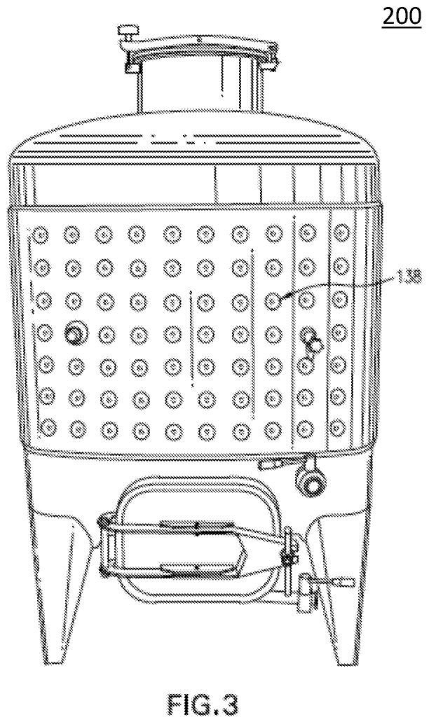

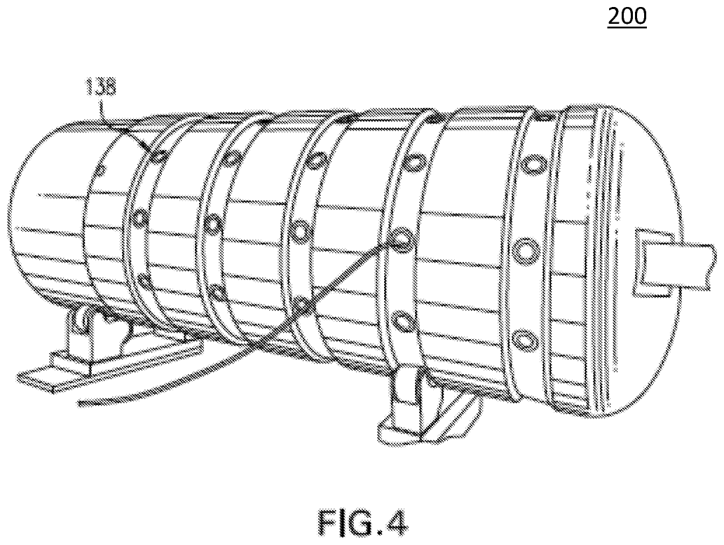

As shown in FIG. 1, a schematic block diagram 100 of a process to create biomass 110 from microalgae 104 is depicted. The microalgae 104 may comprise a photoreceptor sensitive to a region of a visible spectrum. A culture media may comprise a carbon source and may be located inside of a bioreactor, such as a bioreactor 200 of FIG. 2, FIG. 3, and FIG. 4. The microalgae 104 may be located in the culture media. As depicted in FIG. 1, the microalgae 104 may be subjected to numerous conditions, such as light 102, stress conditions 108, and carbon dioxide (CO₂) 106.

The microalgae 104 is configured to convert the atmospheric CO₂ 106 to raw materials (e.g., the biomass 110) via use of the light 102. The biomass 110 may include: proteins, carbohydrates, and lipids and may be directly used for food supplements 112 and/or human food 114, among other uses not explicitly listed herein.

The specific process by which the microalgae 104 converts the atmospheric CO₂₁₀₆ to the raw materials (e.g., the biomass 110) may include a process known as oxygenic photosynthesis. During this process, water is the electron donor, and oxygen is released after hydrolysis. The equation for photosynthesis can be written as follows:

H₂O+CO₂+Photons (light)→[CH₂O]ₙ+O₂ [Equation 1]

The reaction of Equation 1 can be divided into two pathways: (1) a light-dependent reaction and (2) a dark or light-independent reaction. The light-dependent reaction involves both photochemical and redox reaction steps. The overall equation for the light-dependent reaction includes:

2H₂O+2NADP⁺+3ADP+3P+light→2NADPH+2H⁺+3ATP+O₂ [Equation 2]

In this reaction, ADP refers to adenosine diphosphate, P refers to the element phosphate, and NADP refers to nicotinamide adenine dinucleotide phosphate. Light energy is used to synthesize ATP and the NADPH (e.g., the reduced form of NADP⁺), which are energy storage molecules.

In the light-independent reaction, RuBisCO captures CO₂ from the atmosphere. This process requires the newly formed NADPH, called the Calvin cycle or the Benson-Calvin cycle.

The overall equation for the light-independent reaction includes:

3CO₂+9ATP+6NADPH+6H⁺→C₃H₆O₃-phosphate+9ADP+8P+6NADP⁺+3H₂O [Equation 3]

The fixation or reduction of CO₂ takes place by combining CO₂ with a five-carbon sugar, ribulose 1,5-bisphosphate (Ru5BP), generating two molecules of a three-carbon compound, glycerate 3-phosphate (GP). In the presence of ATP and NADPH (from the light-dependent stages), GP is reduced to glyceraldehyde 3-phosphate (G3P) (also called 3-phosphoglyceraldehyde (PGAL) or triose phosphate). Most of the G3P that is produced is used to regenerate Ru5BP so that the process can continue. Of the six molecules of G3P, one is not “recycled” and often condenses to form hexose phosphate, yielding sucrose, starch, and cellulose. The sugars that are produced during carbon metabolism yield carbon skeletons that can be used for other metabolic reactions, such as the production of amino acids and lipids.

The biomass 110 may be converted to biofuels 116 and/or bio-products 118. Examples of the biofuels 116 may include solid biofuels 120, liquid biofuels 122, and/or gas biofuels 124. An example of the solid biofuels 120 may include bio-char. Examples of the liquid biofuels 122 may include bioethanol, biodiesel, vegetable oil, etc. Examples of the gas biofuels 124 may include biohydrogen and/or biosyngas. It should be appreciated that the examples of the biofuels 116 are not limited to those examples explicitly listed herein. Non-exhaustive examples of the bio-products 118 may include: poly-unsaturated fatty acids, antioxidants, coloring agents, vitamins, anti-cancer drugs, anti-microbial drugs, etc.

It should be appreciated that in some examples of the instant disclosure, the process of fermentation of the microalgae 104 may be used to convert algal sugars (such as glucose, fructose, maltose, and/or rhamnose, among other examples not explicitly listed herein) to the liquid biofuels 122 of bioethanol. The yield and quality of the bioethanol (e.g., the liquid biofuels 122) produced is strongly dependent on the fermentation process, which is affected by several factors, such as temperature, pH, oxygen, substrate concentration, and the fermenter organism used. In some examples, the microalgae 104 may be used for food applications. In other examples, the microalgae 104 may be used for pharmaceuticals, antibiotics, plastics replacements, cosmetics, nutritional supplements, flavoring, color pigments, biofuel, cooking oil, etc.

A bioreactor 200 for cultivating microalgae is depicted in FIG. 2, FIG. 3, and FIG. 4. In some examples, the bioreactor 200 may be a photobioreactor. A specific example of the bioreactor 200 is a closed bioreactor, such as a fermentation vessel or tank, as illustrated in FIG. 2, FIG. 3, and FIG. 4. In some examples, the fermentation vessel or tank may be a cylindrical-conical fermentation tank. However, it should be appreciated that the bioreactor 200 may be any type of bioreactor.

The bioreactor 200 may include a glass, a metal, or a plastic tank equipped with gauges and settings to control aeration, stir rate, temperature, pH, and other parameters not explicitly listed herein. Generally the gauges and settings are operatively coupled to the bioreactor 200. In some examples, the bioreactor 200 may be small in size (e.g., 5-10 L or less) to accommodate bench-top applications. In other examples, the bioreactor 200 may be larger in size (e.g., 120,000 L or larger) for use in large-scale industrial applications.

The bioreactor 200 may have one or more ports to allow entry of gases, solids, semi-solids and/or liquids into the chamber containing the microalgae 104. The ports may be attached to tubing or other means of conveying substances. Gas ports, for example, convey gases into the culture media. In some examples, a gas content of the bioreactor 200 may be modified based on the growth of the specific microorganism. In other examples, part of the volume of the bioreactor may contain gas, rather than a liquid. The bioreactor 200 may include gas inlets used to pump gases into the bioreactor 200. Such gases may include air, air/O₂ mixtures, and noble gases, among other gases not explicitly listed herein.

Pumping gases into a bioreactor can feed cells O₂ and other gases, and may also aerate the culture, and therefore, generate turbidity. Increasing gas flow into the bioreactor 200 may increase the turbidity of a culture of the microalgae 104. Placement of ports conveying gases into the bioreactor 200 can also affect the turbidity of a culture at a given gas flow rate. Air/O₂ mixtures can be modulated to generate optimal amounts of O₂ for maximal growth by a particular organism. Microalgae grow significantly faster in the light under, for example, 3% 02/97% air than in 100% air.

Moreover, the bioreactor 200 may have one or more ports that allow media entry and/or for sampling the culture. In some examples, a sampling port can be used repeatedly without altering the axenic nature of the culture. The sampling port can be configured with a valve or other device that allows the flow of sample to be stopped and started. Alternatively, the sampling port can allow continuous sampling.

According to some examples, a bioreactor system for cultivating microalgae is described. The bioreactor system may comprise the bioreactor 200. The bioreactor 200 may comprise one or more holes 138. A culture media may be located inside the bioreactor 200. The culture media may be a liquid medium. In examples, the culture media may contain components such as, a fixed nitrogen source, trace elements, a buffer for pH maintenance, and phosphate. The culture media may also include a fixed carbon source, such as: glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, and/or molasses, among other examples not explicitly listed herein. In additional examples, the carbon source may be preferably glucose. The carbon source can be supplied at a concentration of less than 50 μM, at least about 50 μM, at least about 100 μM, at least about 500 μM, at least about 5 mM, at least about 50 mM, at least about 500 mM, and more than 500 mM of one or more exogenously provided fixed carbon source(s). Additional trace elements may be present in the culture media, such as: zinc, copper, cobalt, boron, manganese, and/or molybdenum.

The microalgae 104 comprising a photoreceptor sensitive to a region of a visible spectrum may be located in the culture media. In some examples, the microalgae 104 is of a mixotrophic strain. In examples, the microalgae 104 may be adapted for both autotrophic growth and heterotrophic growth during a time period. According to some examples, the microalgae 104 may be a Botryococcus sudeticus strain, a Botryococcus strain, a Neochloris oleabundans strain, a Neochloris strain, a Chlamydomonas reinhardtii strain, and/or a Chlamydomonas strain, among other examples not explicitly listed herein.

Exposure of the microalgae 104 to one or more light sources, even in the presence of a fixed carbon source that the cells transport and utilize, can accelerate growth compared to culturing cells of the microalgae 104 in the dark. As such, one or more light sources may be impregnated or implanted into each of the one or more holes 138 of the bioreactor 200. Each of the one or more light sources may produce an irradiance of light in a full spectrum of light. In other examples, each of the one or more light sources may produce the irradiance of the light at a specific wavelength or in a region of the visible spectrum in a sufficient intensity to transduce the photoreceptor of the microalgae 104.

In some examples, each of the one or more light sources may include a natural light source (e.g., sunlight collected by a solar collector and transmitted to the interior of the bioreactor 200 via an optical fiber) or an artificial light source. In other examples, the artificial light source may include a light-emitting diode (LED). In additional examples, the artificial light source may include a fluorescent tube. In some examples, the one or more light sources may include a combination of the natural light source and the artificial light source.

Since uniform distribution of the one or more light sources is essential to avoid photoinhibition, the one or more holes 138 of the bioreactor 200 may be evenly distributed. It should be appreciated that the one or more light sources may be configured to supply a full spectrum or a specific wavelength of artificial light to a bioreactor 200, based on the specific application.

A method executed by the bioreactor system to cultivate the microalgae 104 may grow the microalgae 104 via both light and fermentation. The method may include: incubating the microalgae 104 under the mixotropic growth condition for a time period sufficient to allow the microalgae to grow. The mixotropic growth condition may include the culture media located inside of the bioreactor 200 that comprises the carbon source and an irradiance of light from the one or more light sources that includes the region of the visible spectrum in a sufficient intensity to transduce the photoreceptor. The method may also include producing a material from the microalgae, such as: a polysaccharide, a pigment, a lipid, and/or a hydrocarbon, among other materials. In additional examples, the method may further include: recovering the material and extracting the material.

The material may be harvested, or otherwise collected, by any convenient means. For example, hydrocarbons secreted from cells can be centrifuged to separate the hydrocarbons in a hydrophobic layer from contaminants in an aqueous layer. Extracellular hydrocarbons can also be extracted in vivo from living microalgae cells, which may then be returned to the bioreactor 200 by exposure of the cells to a non-toxic extraction solvent, followed by separation of the living cells and the hydrophobic fraction of extraction solvent and hydrocarbons. The separated living cells are then returned to a culture container. Hydrocarbons can also be isolated by whole cell extraction. Lipids and hydrocarbons generated by the microorganisms of the present invention can be recovered by extraction with an organic solvent. In some cases, the organic solvent is hexane.

The method may further include processing the material, via chemical treatment, to form another material. The other material may include: a fuel, biodiesel, jet fuel, a cosmetic, a pharmaceutical agent, a surfactant, and/or a renewable diesel, among other materials. It should be appreciated that in some examples, the method may also include modifying one or more of the following: modifying the time period to another time period, modifying a pH level of the microalgae 104, modifying a wavelength of the irradiance of the light from the one or more light sources (e.g., modifying the light spectrum) onto the microalgae 104, and/or modifying a feed stock for the microalgae 104 to change a combination of amino acids in the microalgae 104. By doing so, functional proteins can be created.

In another illustrative example, the method may include modifying the wavelength of the irradiance of the light from the one or more light sources onto the microalgae 104 to change the combination of the amino acids in the microalgae 104. In this example, the wavelength of the irradiance of the light from the one or more light sources may include extreme ultraviolet (UV) light. Short exposure of the microalgae 104 to this extreme UV light will modify a genome sequence of the microalgae 104.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A bioreactor system for cultivating microalgae, the bioreactor system comprising:
a bioreactor comprising one or more holes;
a culture media comprising a carbon source and located inside the bioreactor;
a microalgae comprising a photoreceptor sensitive to a region of a visible spectrum, wherein the microalgae is located in the culture media; and
one or more light sources implanted into each of the one or more holes, wherein each of the one or more light sources produce an irradiance of light in the region of the visible spectrum in a sufficient intensity to transduce the photoreceptor of the microalgae.

2. The bioreactor system of claim 1, wherein the microalgae is of a mixotrophic strain.

3. The bioreactor system of claim 2, wherein the microalgae is adapted for autotrophic growth and heterotrophic growth during a time period.

4. The bioreactor system of claim 1, wherein each of the one or more light sources includes an artificial light source.

5. The bioreactor system of claim 1, wherein the artificial light source is a light-emitting diode (LED).

6. The bioreactor system of claim 1, wherein the bioreactor is a fermentation tank.

7. The bioreactor system of claim 1, wherein the microalgae is a strain selected from the group consisting of: a Botryococcus sudeticus strain, a Botryococcus strain, a Neochloris oleabundans strain, a Neochloris strain, a Chlamydomonas reinhardtii strain, and a Chlamydomonas strain.

8. The bioreactor system of claim 1, wherein the carbon source is selected from the group consisting of: glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, and molasses.

9. The bioreactor system of claim 1, wherein the carbon source is glucose.

10. A method executed by a bioreactor system to cultivate a microalgae comprising a photoreceptor sensitive to a region of a visible spectrum under a mixotrophic growth condition, the bioreactor system comprising a bioreactor having one or more light sources implanted into one or more holes of the bioreactor, the method comprising:
incubating the microalgae under the mixotropic growth condition for a time period sufficient to allow the microalgae to grow, wherein the mixotropic growth condition comprises:
a culture media located inside of the bioreactor that comprises a carbon source, and
an irradiance of light from the one or more light sources that includes the region of the visible spectrum in a sufficient intensity to transduce the photoreceptor.

11. The method of claim 10, further comprising:
producing a material from the microalgae.

12. The method of claim 11, wherein the material is selected from the group consisting of: a polysaccharide, a pigment, a lipid, and a hydrocarbon.

13. The method of claim 11, further comprising:
recovering the material; and
extracting the material.

14. The method of claim 13, further comprising:
processing the material to form another material.

15. The method of claim 14, wherein the other material is selected from the group consisting of: a fuel, biodiesel, jet fuel, a cosmetic, a pharmaceutical agent, a surfactant, and a renewable diesel.

16. The method of claim 10, further comprising:
modifying the time period to another time period to change a combination of amino acids in the microalgae.

17. The method of claim 10, further comprising:
modifying a wavelength of the irradiance of the light from the one or more light sources onto the microalgae to change a combination of amino acids in the microalgae.

18. A method executed by a bioreactor system to manufacture a material from a microalgae, the bioreactor system comprising a bioreactor having one or more light sources implanted into one or more holes of the bioreactor, the method comprising:
providing the microalgae comprising a photoreceptor sensitive to a region of a visible spectrum under a mixotrophic growth condition, wherein the microalgae is capable of producing the material;
culturing the microalgae in a media located in the bioreactor, wherein the media comprises a carbon source;
applying an irradiance of light from the one or more light sources including the region of the visible spectrum in a sufficient intensity to transduce the photoreceptor;
producing the material from the microalgae;
recovering the material; and
extracting the material.

19. The method of claim 18, further comprising:
modifying a pH level of the microalgae to change a combination of amino acids in the microalgae.

20. The method of claim 18, further comprising:
modifying a feed stock for the microalgae to change a combination of amino acids in the microalgae.
